(19) United States
(12) Patent Application Publication
(10) Pub. No.: US 2021/0212928 A1
(43) Pub. Date: Jul. 15, 2021

(54) SIRTUIN 1 ACTIVATION AGENT AND SKIN COSMETIC FOR ACTIVATING SIRTUIN 1

(71) Applicant: Bloom Classic Co., Ltd, Tokyo (JP)

(72) Inventors: Hayami TABE, Tokyo (JP); Akihito FUJITA, Tokyo (JP); Mayumi KOTANI, Tokyo (JP); Hidemi SUGIWAKI, Tokyo (JP)

(21) Appl. No.: 17/149,684

(22) Filed: Jan. 14, 2021

(30) Foreign Application Priority Data
Jan. 15, 2020 (JP) 2020004187

(51) Int. Cl. A61K 8/9794; A61K 8/9789; A61Q 19/08

(52) U.S. Cl. CPC A61K 8/9794; A61Q 19/08; A61K 8/9789

(57) ABSTRACT

Provided is a sirtuin-1 activation agent including a natural extract, having high safety as an active ingredient, and a skin cosmetic for activating sirtuin 1. An extract of echinacea and/or banana is used as an active ingredient of the sirtuin-1 activation agent of the present invention. Further, the skin cosmetic for activating sirtuin 1 of the present invention is mixed with the extract of echinacea and/or banana.

### BACKGROUND OF THE INVENTION

### 1. Field of the Invention

The present invention relates to a sirtuin-1 activation agent including an extract of echinacea and/or banana as an active ingredient, and a skin cosmetic for activating sirtuin 1.

### 2. Description of the Related Art

In studies on aging control, it has been found that limiting calories to such a degree that archaebacteria, yeast, nematodes, and humans do not fall into a low-nutrition state provides anti-aging and longevity effects. Sir2 has been identified as one of the molecules involved in these effects. In mammalian homologues, there are seven families of sirtuin. Among them, sirtuin 1, which is most similar in structure and function to Sir2, is attracting attention.

Sirtuin 1 has NAD-dependent deacetylation enzyme activity and ADP ribosyl transferase activity, and plays an important role in living bodies. For example, in the case of mice expressing high levels of sirtuin 1, an improvement in physical ability, prolongation of a reproduction period, and improvements in saccharometabolism, cholesterol metabolism, and fat metabolism have been recognized. Further, improved glucose tolerance and inhibited fatty liver even despite a high-fat diet have been observed. In other words, the activation of sirtuin 1 is believed to be useful for the prevention or treatment of, or recovering from metabolic diseases (Non-Patent Document 1).

Further, the activation of sirtuin 1 enables the p65 subunit of a transcription factor NF-κB (nuclear factor-kappa B) to be deacetylated, and as a result, the NF-κB activity is attenuated, which results in an obvious suppression of inflammation. It is believed that this anti-inflammatory action is capable of being used to prevent or treat, or recover from inflammatory diseases (Non-Patent Document 1). Further, in the mice, it can be seen that the reproductive integrity of the skin is increased by blocking the gene of NF-κB, and the activation of sirtuin 1 is believed to be useful for enhancing the reproductive integrity of the skin (Non-Patent Document 2).

Sirtuin 1 deacetylates FOXO, p53, p73, Ku70, and Smad7, and as a result, induces resistance to oxidative stress and suppresses cell death. It is believed that the induction of phenotypes thereof contributes to the realization of anti-aging and life-prolonging effects (Non-Patent Document 1).

Catabiosis is caused by exposure to extrinsic stress such as ultraviolet light. Catabiosis is a phenomenon in which cell cycles are permanently stopped. It has been found that sirtuin 1 regulates the expression of TERT (telomere reverse transcriptase), so histone is deacetylated, thus maintaining the stability of the telomere, and repair proteins such as WRN protein (Werner syndrome protein) are deacetylated, thus promoting DNA repair, thereby maintaining the stability of the genome, and that these functions inhibit the catabiosis (Non-Patent Document 3).

As described above, it has been found that sirtuin 1 has various functions such as metabolic-disease-recovering action, inflammatory-disease-recovering action, catabiosis-inhibiting action, diabetes-recovering action, cardiovascular-protective action, kidney-disease-recovering action, and neuroprotective action. Therefore, activation of sirtuin 1 is considered to be useful for preventing or treating of, or recovering from various diseases such as metabolic diseases, inflammatory diseases, catabiosis, diabetes, cardiovascular diseases, kidney diseases, and neurological diseases.

As a material for activating sirtuin 1, resveratrol, which is contained in the skin of red grapes in large quantities, is known. Recently, a sirtuin activation agent derived from extracts of black ginger as described in Patent Document 1 has also been reported (Patent Document 1).

### PRIOR ART DOCUMENT

### Patent Document

(Patent Document): Japanese Laid-Open Patent Application No. 2018-199680

### Non-Patent Documents

(Non-Patent Document 1): Chemistry and Biology, 2009, Vol. 47, No. 8, p531-537

(Non-Patent Document 2): Aging cell, 2010, 9, pp285-290

(Non-Patent Document 3): BMB Reports, 2019, 52(1), p. 24-34

### SUMMARY OF THE INVENTION

Accordingly, the present invention has been made keeping in mind the above problems occurring in the related art, and an object of the present invention is to find a material having sirtuin-1-activating action from among natural extracts having high safety and to provide a sirtuin-1 activation agent including the material as an active ingredient and a skin cosmetic for activating sirtuin 1.

In order to accomplish the above object, a sirtuin-1 activation agent of the present invention includes an extract of echinacea and/or banana as an active ingredient. Further, a skin cosmetic for activating sirtuin 1 of the present invention is mixed with an extract of echinacea and/or banana.

A sirtuin-1 activation agent of the present invention includes an extract of echinacea and/or banana, which are natural products, as an active ingredient. Accordingly, it is possible to provide a sirtuin-1 activation agent having not only excellent activity but also high safety.

Further, it is possible to provide a skin cosmetic for activating sirtuin 1, which is mixed with an extract of echinacea and/or banana, thereby having excellent activity and high safety.

### DESCRIPTION OF THE PREFERRED EMBODIMENTS

Hereinafter, an embodiment of the present invention will be described.

[Sirtuin-1 Activation Agent]

A sirtuin-1 activation agent of the present embodiment includes an extract of Echinacea (Scientific name: Echinacea purpurea) and/or banana (Scientific name: Musa Spp.) as an active ingredient.

Examples of an [symbol] extract [symbol] in the present embodiment include an extract solution obtained from Echinacea (Scientific name: Echinacea purpurea) and/or banana (Scientific name: Musa Spp.) as a raw material for extraction, a diluted solution or concentrated solution of the extract solution, a dried substance obtained by drying the extract solution, and a crude-purified substance or purified substance thereof.

Echinacea purpurea is a perennial belonging to the genus Echinacea of the family Asteraceae, and is distributed in North America etc. It is also cultivated for ornamental purposes all over Japan, and can be easily obtained from these regions. Constituent parts of echinacea that can be used as the raw material for extraction include an above-ground part such as a leaf part, a stem part, a flower part, an underground part such as a root part, a seed, a whole plant, or a mixture of these parts, but it is preferably a root part.

Banana (scientific name: Musa spp.) is a plant having an edible fruit among plants belonging to the genus Musa of the family Musaceae, and is cultivated in Southeast Asia, etc., and can be easily obtained from these regions. Although it does not specifically limit as a cultivated variety of banana that can be used as an extraction raw material in the present invention, giant cavendish, Taiwanese banana, grande naine, morado, Shimabanana, Senorita (Monkey Banana), lakata, and plantain can be used as appropriate. Constituent parts of banana that can be used as the raw material for extraction include, for example, an above-ground part such as a leaf part, a pseudostem part, a flower part, a fruit part, a fruit peel part, an underground part such as a stem part, a root part, or a mixture of these parts, but it is preferably a leaf part.

The raw material for extraction may be dried, left or pulverized using a crusher, and may be extracted using an extraction solvent, thus obtaining the extract of the above-mentioned plant. The drying may be performed using sunlight, or may be performed using a dryer that is typically used. Further, pretreatment such as degreasing may be performed using a non-polar extraction solvent such as hexane, thereby efficiently achieving extraction treatment with a polar solvent.

It is preferable to use a polar solvent as the extraction solvent. Examples thereof may include water and a hydrophilic organic solvent, which are preferably used individually or in combination of two or more types thereof at room temperature or at a temperature that is below the boiling point of the solvent.

Examples of the water that may be used as the extraction solvent include purified water, tap water, well water, mineral water, water containing minerals, hot spring water, water that is available for use, and fresh water that has undergone various treatments. Examples of the treatment applied to water include purification, heating, sterilization, filtration, ion exchange, permeation pressure adjustment, and buffering. Therefore, examples of the water that may be used as the extraction solvent in the present embodiment include purified water, hydrothermal water, ion-exchanged water, physiological saline, phosphate buffer, and phosphate-buffered physiological saline.

Examples of the hydrophilic organic solvent that may be used as the extraction solvent may include lower aliphatic alcohols having 1 to 5 carbon atoms such as methanol, ethanol, propyl alcohol, and isopropyl alcohol; lower aliphatic ketones such as acetone and methyl ethyl ketone; and polyhydric alcohols having 2 to 5 carbon atoms, such as 1,3-butylene glycol, propylene glycol, and glycerin.

When a mixed solution of two or more polar solvents is used as the extraction solvent, the mixing ratio thereof may be appropriately adjusted. For example, when a mixed solution of water and lower aliphatic alcohol is used as the extraction solvent, the mixing ratio of the water and the lower aliphatic alcohol is preferably 9:1 to 2:8 (volumetric ratio). Further, when a mixed solution of water and lower aliphatic ketone is used, the mixing ratio of the water and the lower aliphatic ketone is preferably 9:1 to 2:8 (volumetric ratio). Further, when a mixed solution of water and polyhydric alcohol is used, the mixing ratio of the water and the polyhydric alcohol is preferably 9:1 to 1:9 (volumetric ratio), and more preferably 7:3 to 2:8 (volumetric ratio).

The extraction treatment is not particularly limited, as long as the soluble ingredient contained in the raw material for extraction can be eluted in the extraction solvent, and may be performed according to a typical method. For example, the raw material for extraction may be immersed in the extraction solvent in an amount that is 1.5 to 300 times (mass ratio) as large as the amount of the raw material for extraction, and the soluble ingredient may be extracted at room temperature or under reflux by heating and then filtered to remove the extraction residue, thereby obtaining an extract solution. The solvent is distilled from the obtained extract solution to obtain a paste-like concentrate, and the concentrate is further dried, thus obtaining a dried product.

Purification may be performed, for example, using activated carbon treatment, adsorption resin treatment, or ion-exchange resin treatment. The obtained extract solution may be used as an active ingredient of a sirtuin-1 activation agent without further treatment, but the form of a concentrate or dried product is easy to use.

Since the extracts of echinacea and banana obtained as described above have sirtuin-1-activating action, the extracts may be used as the active ingredient of the sirtuin-1 activation agent.

The sirtuin-1 activation agent of the present embodiment may be used for a wide range of applications, such as medicine and medical supplies, quasi-drugs, cosmetics and food products.

The sirtuin-1 activation agent of the present embodiment may include only the extract of echinacea or banana, or may be obtained by formulating the extract of echinacea or banana.

The sirtuin-1 activation agent of the present embodiment may be formulated into any of various kinds of formulations, such as a powder phase, a granular phase, a tablet phase, and a liquid phase, using a pharmaceutically acceptable carrier of dextrin or cyclodextrin and any other auxiliary agent according to a typical method. Examples of the auxiliary agent may include excipients, binders, disintegrants, lubricants, stabilizers, and corrigents. The sirtuin-1 activation agent may be used in combination with other compositions (for example, external preparation for the skin and oral composition etc.), and may be used as ointments, liquid medicines for external use, or sheet agents.

When the sirtuin-1 activation agent of the present embodiment is formulated, the content of the extract of echinacea or banana is not particularly limited, and may be appropriately set according to the purpose.

Meanwhile, the sirtuin-1 activation agent of the present embodiment may be used as an active ingredient by mixing other natural extracts having sirtuin-1-activating action with the extract of echinacea or banana, if necessary.

Examples of a method for administering the sirtuin-1 activation agent of the present embodiment to a patient may include transdermal administration and oral administration, but a method suitable for prevention and treatment may be appropriately selected depending on the type of disease. Further, the dosage of the sirtuin-1 activation agent of the present embodiment may be appropriately increased or decreased depending on the type or severity of disease, individual differences between patients, an administration method, and an administration period.

The sirtuin-1 activation agent of the present embodiment may be used for the purpose of preventing or treating of, or recovering from various diseases such as metabolic diseases, inflammatory diseases, catabiosis, diabetes, cardiovascular diseases, kidney diseases, and neurological diseases, and further for purposes related to various phenomena involving sirtuin 1, such as anti-aging or life-prolonging effects, due to the sirtuin-1-activating action of the extract of echinacea or banana. However, the sirtuin-1 activation agent of the present embodiment may be used for all purposes that have significance in exerting the sirtuin-1-activating action, in addition to the above-described uses.

Extracts of echinacea and banana have sirtuin-1-activating action and also excellent usability or safety when applied to the skin, so the extracts are very suitable for use as an ingredient in an external preparation for the skin in mixing. In the case of the external preparation for the skin, the extract of echinacea or banana may be mixed therein without being treated, or a sirtuin-1 activation agent formulated from the extract of echinacea or banana may be mixed therein. The extract of echinacea or banana or the sirtuin-1 activation agent formulated from the extract of echinacea or banana may be mixed into an external preparation for the skin, thereby imparting sirtuin-1-activating action thereto.

The type of the external preparation for the skin is not limited, and examples thereof include a wide variety of products, such as skin cosmetics as will be described later, and quasi-drugs and medicine and medical supplies which are cutaneously used.

Further, since the extracts of echinacea and banana of the present embodiment have excellent sirtuin-1-activating action, the extracts may be suitably used as a reagent for research on the action mechanisms thereof.

Further, the sirtuin-1 activation agent of the present embodiment is suitable for application to humans, but may be applied to animals other than humans as long as the activity is exhibited.

[Skin Cosmetic for Activating Sirtuin 1]

Since the extracts of echinacea and banana according to the above-described embodiment have excellent sirtuin-1-activating action, the extracts are suitable for mixing with a skin cosmetic. In this case, the extract of echinacea or banana, which is an ingredient for activating sirtuin 1, may be mixed without any treatment, or the sirtuin-1 activation agent formulated from the extract of echinacea or banana may be mixed.

The kind of skin cosmetic that may be mixed with the extract of echinacea or banana is not particularly limited, and examples thereof may include ointments, creams, milky lotions, lotions, packs, and foundations.

When the extract of echinacea or banana is mixed with the skin cosmetic, the amount thereof that is mixed therein may be appropriately adjusted depending on the type of the skin cosmetic. However, a suitable mixing ratio is 0.0001 to 10 mass % (in terms of solid content), and a particularly suitable mixing ratio is 0.001 to 1 mass % (in terms of solid content).

As long as the sirtuin-1-activating action of the extracts of echinacea and banana is not impeded, the skin cosmetic of the present embodiment may be used in combination with main agents, auxiliary agents, or other ingredients, for example, astringents, germicides/antimicrobials, UV absorbers, moisturizers, cell potentiators, anti-inflammatory agents/antiallergic drugs, antioxidants/active-oxygen removing agents, fats and oils, waxes, hydrocarbons, fatty acids, alcohols, esters, surfactants, and flavorings, which are used in the manufacture of typical skin cosmetics. By using them together in this way, the skin cosmetic becomes a more general product, and the synergistic action between the above-mentioned ingredients used in combination therewith results in usage effects better than those typically expected.

The skin cosmetic has high safety, and may be used for the purpose of preventing or treating of, or recovering from various diseases such as metabolic diseases, inflammatory diseases, catabiosis, diabetes, cardiovascular diseases, kidney diseases, and neurological diseases, and the further purpose of various phenomena involving sirtuin 1, such as anti-aging and life-prolonging effects, due to the sirtuin-1-activating action thereof.

[Oral Composition for Activating Sirtuin 1]

The extracts of echinacea and banana have sirtuin-1-activating action and also excellent safety, so the extract is suitable for mixing with an oral composition. In the case of the oral composition, the extract of echinacea or banana may be mixed without any treatment, or a sirtuin-1 activation agent formulated from the extract of echinacea or banana may be mixed. The extract of echinacea or banana or the sirtuin-1 activation agent formulated from the extract of echinacea or banana may be mixed to enable the oral composition to have sirtuin-1-activating action.

The oral composition means a product which is not likely to harm human health and which is taken by oral or digestive-tract administration in normal social life. The food product is not limited to categories of foods, medicine and medical supplies, and quasi-drugs according to the administrative classification. Therefore, the term ┌oral composition┘ in the present embodiment includes a wide variety of products, such as general foods, health foods, health functional foods (specific health foods, nutritive functional foods, foods with function claims), quasi-drugs, and medicine and medical supplies that are orally taken.

When the extract of echinacea or banana is mixed with the oral composition, the mixing amount of the active ingredient thereof may appropriately vary depending on the purpose of use, symptoms, and gender. However, in consideration of the general intake amount of the oral composition to be added, it is preferable that the intake amount of the extract per adult be about 1 to 1000 mg per day. Further, when the oral composition to be added is a granular-, tablet-, or capsule-type oral composition, the amount of the extract of echinacea or banana that is added is typically 0.1 to 100 mass %, and preferably 5 to 100 mass %, based on the amount of the oral composition to be added.

The oral composition of the present embodiment may be mixed with any oral composition that does not interfere with the activity of the extract of echinacea or banana, or may be a nutritional supplement including the extract of echinacea or banana as a main ingredient.

When the oral composition of the present embodiment is manufactured, for example, an arbitrary auxiliary agent, for example, sugars such as dextrin or starch, proteins such as gelatin, soy proteins, or corn proteins, amino acids such as alanine, glutamine, or isoleucine, polysaccharides such as cellulose or Arabic gum, and fats and oils such as soybean oil or medium-chain fatty acid triglycerides, may be added thereto to thus obtain a predetermined form of oral composition.

The oral composition that may be mixed with the extract of echinacea or banana is not particularly limited. However, specific examples thereof may include beverages such as soft drinks, carbonated drinks, nutritious drinks, fruit drinks, and lactic acid drinks (including the concentrated crude liquid of the drink and powder for adjustment); ice cakes such as ice creams, ice sorbets, and ice flakes; noodles such as buckwheat noodles, udon noodles, cellophane noodles, dumpling wraps, Xiaomi wraps, Chinese noodles, and instant noodles; confectionery such as Korean hard taffy, chewing gum, candy, gum, chocolate, Jeongkwa, snack cookies, biscuits, jellies, jams, creams, and baked cookies; processed seafood and livestock foods such as fish cakes, ham, and sausage; dairy products such as processed milk and fermented milk; fats and oils and processed fat-and-oil-based foods such as salad oil, frying oil, margarine, mayonnaise, shortening, whipping cream, and dressing; seasonings such as sauces and marinades; soups, stews, salads, side dishes, pickles, various other health and nutritional supplement foods, tablets, capsules, and health drinks. When the extract of echinacea or banana is mixed with the oral composition, auxiliary raw materials or additives that are commonly used may be also used.

### EXAMPLES

Hereinafter, the present invention will be specifically described with reference to Examples, but the present invention is not limited to the following Examples.

### Example 1

### Preparation of Extract Solution of a Root Part of Echinacea

4 kg of 50% BG (1,3 butylene glycol, Haisugarcane B G, manufactured by Kokyu Alcohol Kogyo Co., Ltd) was mixed with 20 g of the dried product of the root part of Echinacea purpurea and immersed for 7 days. Filtering was performed using ADVANTEC qualitative filter paper (No.2, manufactured by Toyo Roshi Kaisha, Ltd) and a membrane (0.45 μm), and an extract of the root part of Echinacea (solid content: 3.19 mass %) was obtained as a filtrate.

### Example 2

### Preparation of Extract Solution of a Leaf Part of Banana

20 g of dried leaves of banana (Musa Spp., Shima Banana) were mixed with 4 kg of 50% BG (1,3-butylene glycol, Haisugacane B G, manufactured by Kokyu Alcohol Kogyo Co., Ltd), and then immersed therein for seven days. Filtration was performed using an ADVANTEC qualitative filter paper (No. 2, manufactured by Toyo Roshi Kaisha, Ltd) and a membrane (0.45 μm), and the extract (solid content: 0.33 mass %) of the banana leaf part was obtained as a filtrate.

### Comparative Example 1

### Preparation of NMN (Nicotine Amide Mononucleotide) Solution

NMN (nicotine amide mononucleotide) was purchased from Tokyo Chemical Industry Co., Ltd., and a phosphate buffer solution (PBS(1), manufactured by Sigma-Aldrich Co. LLC.) was used to prepare a 0.43% NMN solution.

### Test Example 1

### Expression Test of Sirtuin 1 Gene

The expression test of the sirtuin 1 gene was performed on the extract solution of the root part of Echinacea that was pruned in Example 1, the extract solution of the leaf part of banana of Example 2, and the NMN solution of Comparative Example 1 as follows.

Human neonatal foreskin-derived fibroblasts (number of succeeded generations was 3) were pre-cultured under 5% CO₂ at 37° C. using Dulbecco's Modified Eagle Medium (DMEM, manufactured by Thermo Fisher Scientific Inc.) containing 10% fetal bovine serum (FBS, manufactured by Thermo Fisher Scientific Inc.), and cells were collected by treatment with 0.25% trypsin (manufactured by Sigma-Aldrich Co. LLC.). The recovered cells were diluted with 10% FBS/DMEM at a concentration of 3×10⁴ cells/ml and then sown in an amount of 5 mL in each of fifty flasks for culturing cells (manufactured by Sumitomo Bakelite Co., Ltd.), followed by culturing for 2 hours. Thereafter, 1.35 μL of Example 1 (sample concentration of 8.6 μg/mL), 13 μL of Example 2 (sample concentration of 8.6 μg/mL), and 10 μL (sample concentration of 8.6 μg/mL) of Comparative Example 1 were added thereto, followed by culturing for three days.

After confirming that there was no cytotoxicity, the culture medium was removed, washing was performed with a phosphate buffer solution (PBS(1), manufactured by Sigma-Aldrich Co. LLC.), and 1 mL of a 1% SDS solution (manufactured by NIPPON GENE CO., LTD.) was added thereto to thus recover cells. After the cell suspension was sufficiently agitated using a vortex, 180 μL of the cell suspension was sampled. 1 μL, of 1% KOH (manufactured by NACALAI TESQUE, INC.) and 20 μL of a proteinase K solution (manufactured by Thermo Fisher Scientific Inc.) were added thereto and then agitated, followed by incubation at 37° C. for 15 minutes. After incubation, 100 μL of RNA Clean XP (manufactured by Beckman Coulter, Inc.) was added to each, agitated, and left on a magnet stand for 5 minutes. The supematant was removed, washing was performed twice using 85% ethanol, drying was performed for 10 minutes, and 30 μL of nuclease-free water (manufactured by Thermo Fisher Scientific Inc.) was added thereto. The resultant substance was left on a magnet stand for 5 minutes, and supematant treatment was performed using an RNA solution.

Preparation was performed so that the ratio of Super Script™ IV VILO™ Master Mix (manufactured by Thermo Fisher Scientific Inc.) to nuclease-free water was 1:2.5 in a 200-μL PCR tube (manufactured by Bio-Rad laboratories Inc., clear, dome cap) at 0° C., followed by agitation using a vortex and dispensing in an amount of 14.0 μL in another PCR tube. An NRT sample was prepared so that the ratio of Super Script™ W VILO™ No RT Control (manufactured by Thermo Fisher Scientific Inc.) to nuclease-free water was 1:2.5, followed by dispensing. The RNA solution obtained as described above was added in an amount of 6.0 μL thereto, followed by incubation using a thermal cycler (manufactured by Bio-Rad laboratories Inc., T100™ Thermal cycler) at 25° C. for 10 minutes, at 50° C. for 10 minutes, and at 85° C. for 5 minutes, thereby obtaining a cDNA sample and an NRT sample.

Preparation was performed so that the ratio of Taqman^{(R)} Gene Expression Assay (ACTB Hs99999903_ml or SIRT1 Hs01009006_ml, manufactured by Thermo Fisher Scientific Inc.), TaqPath™ qPCR Master Mix, CG (manufactured by Thermo Fisher Scientific Inc.), and nuclease-free water was 1:10:5. The resultant sample was placed in a nuclease-free tube, agitated using a vortex, and spun down. The resultant substance was dispensed in an amount of 16.0 μL in a PCR tube (manufactured by Bio-Rad laboratories Inc., white, flat cap), and a cDNA sample and an NRT sample were each added in amounts of 4.0 μL, and pipetting and agitation using a vortex were performed, followed by spinning down.

Real-time PCR (manufactured by Bio-Rad laboratories Inc., C1000 Touch™ Thermal Cycler) was performed using the above-described samples. The PCR was performed under PCR conditions including 25° C. for 2 minutes, 95° C. for 20 seconds, 95° C. for 3 seconds (1), and 60° C. for 30 seconds (2) (1→2 40 cycles).

The results are set forth in Table 1.

TABLE 1

| | Sample | | | | | |
|---|---|---|---|---|---|---|
| | concentration | Sirtum 1 Cq | β-actin Cq | ΔCq | Ratio *1000 | Relative ratio |
| Control untreated | 0 μg/mL | 27.53 | 20.72 | 6.81 | 8.93 | 1 |
| Example 1 | 8.6 μg/mL | 28.17 | 22.54 | 5.63 | 20.17 | 2.26 |
| Example 2 | 8.6 μg/mL | 27.61 | 21.93 | 5.68 | 19.54 | 2.19 |
| Comparative | 8.6 μg/mL | 28.53 | 21.42 | 7.11 | 7.24 | 0.81 |
| Example 1 | | | | | | |

Cq: Number of cycles at which an amplification curve and a critical value cross
β-actin: Implicit control
ΔCq: Sirtuin Cq − β-actin Cq
Ratio * 1000: 2^{(-ΔCq)} × 1000

When a sample concentration was 8.6 μg/mL, the expression of the sirtuin 1 gene was recognized to be 2.26 times higher in Example 1 than in an untreated control and 2.19 times higher in Example 2 than in the untreated control, but expression of the sirtuin 1 gene was not observed at the same concentration in Comparative Example 1. From the above results, the activity of the sirtuin 1 gene was recognized to be stronger in Examples 1 and 2 than in Comparative Example 1.

### Mixing Example 1

Milky lotion was manufactured according to the following composition using a typical method.

| | |
|---|---|
| echinacea extract | 0.01 g |
| Jojoba oil | 4.00 g |
| 1,3-butylene glycol | 3.00 g |
| Arbutin | 3.00 g |
| Polyoxyethylene cetyl ether (20E.0.) | 2.50 g |
| Olive oil | 2.00 g |
| Squalene | 2.00 g |
| Cetanol | 2.00 g |
| Glyceryl monostearate | 2.00 g |
| Polyoxyethylene sorbitan oleate (20E.0.) | 2.00 g |
| Methyl parahydroxybenzoate | 0.15 g |
| Stearyl glycyrrhetinate | 0.10 g |
| Extract of milk vetch root | 0.10 g |
| Dipotassium glycyrrhizate | 0.10 g |
| Ginkgo leaf extract | 0.10 g |
| Conchiolin | 0.10 g |
| Amur cork tree extract | 0.10 g |
| Chamomile extract | 0.10 g |
| Flavoring | 0.05 g |
| Purified water | Balance (total amount |
| | is 100 g) |

### Mixing Example 2

Creams having the following composition were manufactured using a typical method.

| | |
|---|---|
| banana extract | 0.01 g |
| Sophorae radix extract | 0.1 g |
| Turmeric extract | 0.1 g |
| Liquid paraffin | 5.0 g |
| White beeswax | 4.0 g |
| Squalene | 10.0 g |
| Cetanol | 3.0 g |
| Lanolin | 2.0 g |
| Stearic acid | 1.0 g |
| Polyoxyethylene sorbitan oleate (20E.0.) | 1.5 g |
| Glyceryl monostearate | 3.0 g |
| Oil-soluble licorice extract | 0.1 g |
| 1,3-butylene glycol | 6.0 g |
| Methyl parahydroxybenzoate | 1.5 g |
| Flavoring | 0.1 g |
| Purified water | Balance (total amount |
| | is 100 g) |

### Mixing Example 3

A cosmetic liquid having the following composition was manufactured using a typical method.

| | |
|---|---|
| echinacea extract | 0.01 g |
| banana extract | 0.01 g |
| Ascorbic acid 2-glucoside | 0.1 g |
| Chamomile extract | 0.1 g |
| Carrot extract | 0.1 g |
| Xanthan gum | 0.3 g |
| Hydroxyethyl cellulose | 0.1 g |
| Carboxyvinyl polymer | 0.1 g |
| 1,3-butylene glycol | 4.0 g |
| Dipotassium glycyrrhizate | 0.1 g |
| Glycerin | 2.0 g |
| Potassium hydroxide | 0.25 g |
| Flavoring | 0.01 g |
| Preservative (methyl parahydroxybenzoate) | 0.15 g |
| Ethanol | 2.0 g |
| Purified water | Balance (total amount |
| | is 100 g) |

The sirtuin activation agent of the present invention may greatly contribute to the realization of prevention or treatment of, or recovery from, various diseases, such as metabolic diseases, inflammatory diseases, catabiosis, diabetes, cardiovascular diseases, kidney diseases, and neurological diseases, and the realization of anti-aging and life-prolonging effects.

What is claimed is:

1. A sirtuin-1 activation agent comprising:
an extract of echinacea and/or banana as an active ingredient.

2. The activation agent of claim 1, the extract of echinacea or banana increases activation of sirtuin-1 gene two times or more.

3. The activation agent of claim 1, a concentration of the extract of echinacea or banana required for activation of sirtuin-1 gene is lower than a concentration of NMN(Nicotine amide mononucleotide) solution required for activation of the sirtuin-1 gene.

4. The activation agent of claim 1, the extract of echinacea is an extract extracted from a root part of echinacea.

5. The activation agent of claim 1, the extract of banana is an extract extracted from a leaf part of banana

6. A skin cosmetic for activating sirtuin 1, which is mixed with an extract of echinacea and/or banana.

7. The skin cosmetic of claim 6, the extract of echinacea or banana increases activation of sirtuin-1 gene two times or more.

8. The skin cosmetic of claim 6, a concentration of the extract of echinacea or banana required for activation of sirtuin-1 gene is lower than a concentration of NMN(Nicotine amide mononucleotide) solution required for activation of the sirtuin-1 gene.

9. The skin cosmetic of claim 6, the extract of echinacea is an extract extracted from a root part of echinacea.

10. The skin cosmetic of claim 6, the extract of banana is an extract extracted from a leaf part of banana.
